(19) United States
(12) Patent Application Publication
(10) Pub. No.: US 2022/0354707 A1
(43) Pub. Date: Nov. 10, 2022

(54) MANIPULATABLE WITHDRAWAL STRINGS FOR TAMPONS

(71) Applicant: Flextring Ltd, Haifa (IL)

(72) Inventors: Maya LINCOLN-WASSER, Haifa (IL); Shlomo Abraham Avi WASSER, Haifa (IL)

(73) Assignee: Flextring Ltd, Haifa (IL)

(21) Appl. No.: 17/769,749

(22) PCT Filed: Oct. 15, 2020
(86) PCT No.: PCT/IL2020/051096

§ 371 Date: Apr. 17, 2022

Related U.S. Application Data
(60) Provisional application No. 62/916,264, filed on Oct. 17, 2019.

(51) Int. Cl. A61F 13/34; A61F 13/20

(52) U.S. Cl. CPC A61F 13/34; A61F 13/2022

(57) ABSTRACT

A tampon apparatus including a tampon having distal, insertion end and a proximal end, and a tampon-withdrawal-string having a tied end and a free end. The tied end of the tampon-withdrawal-string is securely attached to the proximal end of the tampon, At least a portion of the tampon-withdrawal-string is a devised-portion, wherein the devised-portion is either elastic or pliant. While the tampon is in an inserted position, the devised-portion facilitates the user to manipulate the tampon-withdrawal-string or a portion thereof. Typically, the free end of the tampon-withdrawal-string is comfortably reachable to a user when the tampon is in an inserted position.

### FIELD OF THE INVENTION

The disclosure relates to a tampon apparatus having a tampon body and a pulling string fastened onto one end of the tampon body and more specifically, to manipulatable withdrawal strings for tampon apparatuses having a rest, compact form and a pulled, straightened form.

### BACKGROUND

A tampon, in particular a tampon for feminine hygiene, is typically provided with a withdrawal string. The withdrawal string enables to easily pull out and remove the tampon from the user's vaginal opening.

One of the drawbacks related to a. common tampon-withdrawal-suing is that the string may get wet and dirty during usage, e.g. while urinating, defecating or wiping after defecation and also as a result of its proximity to the internal part of the toilet, that might contain pee, feces, and toilet water. A wet and dirty withdrawal string wets underwear and cloths, and makes the tampon uncomfortable to wear, and is also a source of infection.

Another drawback related to a common tampon-withdrawal-string is that the withdrawal string might also be visible through different outfits (e.g. short trousers, swimming suits, semitransparent cloths, dancing cloths, etc.), or when the woman is naked. Such a visible string is a source of embarrassment for some woman.

Therefore, there is a need for tampons having a short withdrawal string that will be invisible as much as possible. On the other hand, a withdrawal string with a normal length (typically, with no limitations, 10 to 15 cm) is considered by most women as more comfortable for the purpose of gripping and pulling the tampon out of the vaginal opening. Thus, the string has to be fairly long to allow the fingers to get a solid grip on the string before pulling to remove the tampon. Therefore, there is a need for a short withdrawal string that is also convenient for handling.

Some prior art publications deal with the withdrawal string length issue and others suggest means for solving one or more of the above problems as follows: US Patent application US/2007/1919805 specifies a tampon which allows a user to change the effective length of the withdrawal string before or during usage. This suggested withdrawal string configuration requires an additional activity to the usual process of handling a tampon (extending/shortening the string), which might be cumbersome for some women.

U.S. Pat. No. 6,312,419 deals with the problem that the standard length of the withdrawal string of the tampon is considered too short. This patent suggests a lengthening device which can be added to the withdrawal string in order to improve its handling and usage.

U.S. Pat. No. 6,142,489 deals with the problem that the withdrawal string sometimes gets saturated with liquid as a result of urination. This patent suggests a coating for the withdrawal string, This specification, however, does not deal with the length of the withdrawal string.

U.S. Pat. No. 2,587,515 suggests a coil at the loose end of the withdrawal string of the tampon for improving the packaging process of tampons: This patent is not aimed at shortening and hiding the withdrawal string, and the coil is not intended to return to its original compressed state after opening the package and during usage.

U.S. Pat. No. 3,037,506 suggests an extensible elastic withdrawal string aimed at causing a small absorbent pad to springingly engage against the vaginal opening. One of the suggested configurations in the current patent, as detailed below, also includes an extensible elastic withdrawal string, but the string of the present disclosure differentiates from the string in U.S. Pat. No. 3,037,506 in the following ways: (1) the string of the present disclosure is aimed at shortening the withdrawal string; (2) in preferred embodiments the string of the present disclosure is not used for pulling any object against the vaginal opening; and (3) in preferred embodiments the string of the present disclosure is not connected to any absorbent pad.

The following patents also aim at shortening and/or hiding the tampon-withdrawal-string in the following ways:

According to FR Patent publication 2,345,989 the tampon-withdrawal-string is made short enough to remain within the vaginal lips, or alternatively, not to protrude by>4 cm. In another configuration the string is long and is rolled up and secured at the end of the tampon, to be undone when required.

DE utility model 9,011,100 suggests applying a color coating to the tampon-withdrawal-string that seems like the natural body hair in order to hide the withdrawal string.

JP utility model JP3033710U and JP Patent application 2006/271919A suggests a tampon-withdrawal-string that looks like a pubic curl, in order to camouflage the string's appearance.

U.S. Pat. No. 9,566,194 proposes a withdrawal string for a tampon in the shape of a loop in order to avoid problems encountered with long strings because the loop is preferably short enough that it will not accidently be exposed from a garment and stay out of the way of the urine stream.

U.S. Pat. No. 9,744,081 also proposes a withdrawal string for a tampon in the shape of a loop in order to shorten the withdrawal string. In the present disclosure one end of the loop can be removed to make the cord longer.

The present disclosure aims at providing a solution to prevent the exposure of a. tampon-withdrawal-string, while maintaining the safe, reliable and convenient usage of tampon withdrawal strings. Another intention of the present disclosure is to maintain the tampon-withdrawal-string close as much as possible to the user's body and distant as much as possible from wetness/dirt sources in order to avoid situations in which the string gets wet and dirty (e.g. while urinating, defecating or wiping after defecation, and also as a result of its proximity to the internal part of the toilet).

### SUMMARY

An embodiment is an example or implementation of the present disclosures. The various appearances of “one embodiment,” “an embodiment” or “some embodiments” do not necessarily all refer to the same embodiment. Although various aspects of the present disclosure may be described in the context of a single embodiment, the aspects may also be provided separately or in any suitable combination. Conversely, although the disclosure may be described herein in the context of separate embodiments for clarity, the present disclosure may also be implemented in a single embodiment.

Reference in the specification to “one embodiment”, “an embodiment”, “some embodiments” or “other embodiments” means that a particular feature, structure, characteristic, or other aspects described in connection with the embodiments is included in at least one embodiment, but not necessarily all embodiments, of the present disclosure. It is understood that the phraseology and terminology employed herein are not to be construed as limiting and are for descriptive purpose only.

According to the teachings of the present disclosure there is provided a tampon apparatus including a tampon having distal, insertion end and a proximal end, and a tampon-withdrawal-string having a tied end and a free end, wherein the tied end of the tampon-withdrawal-string is securely attached to the proximal end of the tampon. At least a portion of the tampon-withdrawal-string is a devised-portion, wherein the devised-portion is either elastic or pliant. While the tampon is in an inserted position, the devised-portion facilitates the user to manipulate the tampon-withdrawal-string or a portion thereof.

Typically, the free end of the tampon-withdrawal-string is comfortably reachable to a user when the tampon is in an inserted position.

The manipulating of the tampon-withdrawal-string includes activities selected from the group including:

a. maintaining the tampon-withdrawal-string less-visible;

b. maintaining the tampon-withdrawal-string in a comfort position;

c. maintaining the tampon-withdrawal-string in a position far from wetness/dirt sources, thereby making the tampon-withdrawal-string more hygienic;

d. straitening the tampon withdrawal string; and

e. pulling out and rearranging portions of the tampon-withdrawal-string, with or without pulling the tampon from the inserted position.

While the tampon is in an inserted position, the devised-portion may extend to a maximum length of Ds when pulling the tampon-withdrawal-string with a force Fin Fs, without pulling the tampon from the inserted position, and when pulling the tampon-withdrawal-string with a force F_{P2}>F_{S}, at some point the tampon pulls out of the vaginal opening.

In one embodiment of the present disclosure, the devised-portion is elastic.

Typically, the elastic devised-portion, when in compact form, has dimensions that keep the tampon-withdrawal-string close to the user body and thereby hidden from potential observers.

Typically, the elastic devised-portion, when in compact form, has dimensions that keep the tampon-withdrawal-string away from the anus and the urethral opening to thereby keep the tampon-withdrawal-string cleaner and drier during usage.

Optionally, the elastic devised-portion reaches either the free end of the tampon-withdrawal-string or proximal thereto, or reaches the proximal end of the tampon or proximal thereto.

Optionally, the elastic devised-portion reaches both the free end of the tampon-withdrawal-string, or proximal thereto, and the proximal end of the tampon, or proximal thereto.

Optionally, the elastic devised-portion does not reach both the free end of the tampon-withdrawal-string, or proximal thereto, and the proximal end of the tampon, or proximal thereto,

Optionally, the elastic devised-portion is arranged in a one-dimensional (1D) form or a two-dimensional (2D) form or a three-dimensional (3D) form.

Optionally, the elastic devised-portion is made from an elastic material, wherein the elastic material is selected from a group of materials including silicon and polyester.

Optionally, the elastic devised-portion is semi-rigid, wherein the semi-rigid elastic devised-portion may include a common, non-elastic string coated with a semi rigid material selected from the group of materials including silicon, and polyester.

Optionally, the elastic devised-portion is made from a knitted yarn.

Optionally, the elastic devised-portion elasticity is formed by applying manufacturing methods selected from the group of methods including:

a. twisting yarn;

b. knitting the yarn in a compact and elastic form with memory like a spiral shape, a zigzag, or as a skein;

c. shaping the yarn in a compact form and applying different types of coating to the yarn;

d. using an elastomeric thread as the core, wherein the core is wrapped with a non-elastic textile fiber: and

e. producing the yarn using the pneumatic looping technique.

Optionally, the elastic devised-portion ends with a pulling tail.

Optionally, the pulling tail of the elastic devised-portion is non-elastic.

Optionally, the pulling tail of the elastic devised-portion is pliant.

Optionally, the elastic devised-portion is arranged as a 3D coil, wherein the 3D coil may be a compact coil.

Optionally, the elastic devised-portion is arranged in a 3D skein form.

Optionally, the elastic devised-portion is arranged in a 2D form and selected from the group of 2D shapes including one or more closed rounded loops, one or more open rounded loops, one or more rhombuses and a zigzag.

In other embodiments of the present disclosure, the devised-portion is pliant.

Preferably, manipulating of the pliant tampon-withdrawal-string includes shaping the tampon-withdrawal-string in a desired form.

Optionally, the pliant devised-portion reaches a pulling tail, wherein the pulling tail is either non-elastic or elastic.

Optionally the pliant devised-portion reaches either the free end of the tampon-withdrawal-string or proximal thereto, or reaches the proximal end of the tampon or proximal thereto.

Optionally, the pliant devised-portion reaches both the free end of the tampon-withdrawal-string (or proximal thereto and the proximal end of the tampon (or proximal thereto).

Optionally, the pliant devised-portion does not reach both the free end of the tampon-withdrawal-string, or proximal thereto, and the proximal end of the tampon, or proximal thereto.

According to further teachings of the present disclosure there is provided a method for enhancing a tampon apparatus user's ability to place and manipulate a tampon-withdrawal-string that is secured to a tampon of a tampon apparatus, during usage by a user, the method including the steps of providing the tampon-withdrawal-string with a devised-portion facilitating the user to manipulate the tampon-withdrawal-string or a portion thereof, wherein the devised-portion is either elastic or pliant.

Preferably, when the devised-portion of the method is placed in a rest state and not manipulated, the tampon-withdrawal-string is kept hidden from potential observers.

Preferably, when the devised-portion of the method is placed in a rest state and not manipulated, the tampon-withdrawal-string is kept away from the urethral opening and the anus to thereby keep the tampon-withdrawal-string cleaner and drier during usage.

Optionally, the devised-portion of the method is elastic.

Optionally, when in compact form, the elastic devised-portion of the method has dimensions that keep the tampon-withdrawal-string close to the user body and thereby hidden from potential Observers.

Optionally, when in compact form, the elastic devised-portion of the method, has dimensions that keep the tampon-withdrawal-string away from the anus and the urethral opening, to thereby keep the tampon-withdrawal-string cleaner and drier during usage.

Optionally, when the tampon apparatus of the method is in use and the elastic devised-portion is pulled outwardly to a straightened form, the length of the straightened elastic devised-portion provides a comfortable length for the user to comfortably pull out the tampon apparatus.

Optionally, the devised-portion of the method is pliant, and the method further includes the step of shaping the pliant devised-portion of the tampon-withdrawal-string.

Optionally, the method further includes the step of placing the pliant devised-portion in different locations, for example with no limitations, distal from the urethral opening and the anus.

Optionally, when the tampon apparatus of the method is in use and the pliant devised-portion is shaped to a straightened form, the length of the straightened pliant devised-portion provides a comfortable length for the user to comfortably pull out the tampon apparatus.

Optionally, the method further includes the step of shaping and placing the pliant devised-portion of the tampon-withdrawal-string in a comfortable way, including adapting the pliant devised-portion to the anatomy of the user.

### BRIEF DESCRIPTION OF THE DRAWINGS

The present disclosure is herein described, by way of non-limiting example, with reference to the accompanying drawings. With specific reference now to the drawings in detail, it is stressed that the particulars shown are by way of example and for purposes of illustrative discussion of the embodiments of the present disclosure only, and are presented in order to provide what is believed to be the most useful and readily understood description of the principles and conceptual aspects of the present disclosure. In this regard, no attempt is made to show structural details of the present disclosure in more detail than is necessary for a fundamental understanding of the present disclosure, the description taken with the drawings making apparent to those skilled in the art how the several forms of the present disclosure may be embodied in practice. In the figures:

FIG. 1a schematically illustrates an example tampon apparatus having a tampon-withdrawal-string with a three-dimensional (3D), elastic compact coiled string portion, according to aspects of the present disclosure, wherein the tampon-withdrawal-string is in a rest state.

FIG. 1b schematically illustrates an example tampon apparatus as in FIG. 1a., wherein the tampon-withdrawal-string is in a rest state and wherein the coil ends with a pulling tail.

FIG. 1b′ schematically illustrates an example tampon apparatus as in FIG. 1b, wherein the tampon-withdrawal-string is in a straightened state, for example, while pulling the tampon out of the vaginal opening.

FIG. 1c schematically illustrates another example tampon apparatus having an elastic 3D helical tampon-withdrawal-string, according to aspects of the present disclosure, wherein the tampon-withdrawal-string is in a rest state.

FIG. 1d schematically illustrates an example tampon apparatus having a tampon-withdrawal-string with a non-elastic segment and an elastic 3D helical segment, according to aspects of the present disclosure, wherein the tampon-withdrawal-string is in a rest state.

FIG. 1d′ schematically illustrates an example tampon apparatus as in FIG. 1d, wherein the tampon-withdrawal-string is in a straightened state.

FIG. 1e schematically illustrates another example tampon apparatus having a tampon-withdrawal-string with two non-elastic segments and an elastic 3D skein segment, according to aspects of the present disclosure, wherein the tampon-withdrawal-string is in a rest state.

FIG. 2a schematically illustrates an example tampon apparatus having a tampon-withdrawal-string with an elastic two-dimensional (2D) rhombus form, according to aspects of the present disclosure, wherein the tampon-withdrawal-string is in a rest state.

FIG. 2b schematically illustrates another example tampon apparatus having a tampon-withdrawal-string with an elastic 2D rounded loop form, according to aspects of the present disclosure, wherein the tampon-withdrawal-string is in a rest state.

FIG. 2c schematically illustrates another example tampon apparatus having a tampon-withdrawal-string with two non-elastic segments and a compact segment shaped in an elastic wide 2D zigzag form with rounded corners, according to aspects of the present disclosure, wherein the tampon-withdrawal-string is in a rest state.

FIG. 2d schematically illustrates more example tampon-withdrawal-strings for a tampon having various elastic 2D shapes in a rest state, according to aspects of the present disclosure.

FIG. 2e schematically illustrates an example tampon apparatus having a tampon-withdrawal-string with two non-elastic segments and a row of 3 elastic rounded loop forms, wherein the tampon-withdrawal-string is in a rest state, according to aspects of the present disclosure.

FIG. 2e′ schematically illustrates an example tampon apparatus as in FIG. 2e, wherein the tampon-withdrawal-string is in a straightened state,

FIG. 3 schematically illustrates an example tampon apparatus having an elastic tampon-withdrawal-string, according to aspects of the present disclosure, wherein the solid line illustrates an elastic tampon-withdrawal-string in a rest state and the unification of the solid and segmented lines illustrates the extended tampon-withdrawal-string up to the maximum length of the straightened state.

FIG. 4 schematically illustrates an example tampon apparatus having a twisted tampon-withdrawal-string, according to aspects of the present disclosure, wherein the tampon-withdrawal-string is in a rest state,

FIG. 5 schematically illustrates an example tampon apparatus having a pliant withdrawal string, according to aspects of the present disclosure, wherein the tampon-withdrawal-string is in an example rest state.

### DETAILED DESCRIPTION

The present disclosure will now be described more fully hereinafter with reference to the accompanying drawings, in which preferred embodiments of the present disclosure are shown. This disclosure may, however, be embodied in many different forms and should not be construed as limited to the embodiments set forth herein; rather, these embodiments are provided, so that this disclosure will be thorough and complete, and will fully convey the scope of the present disclosure to those of ordinary skill in the art.

Various modifications to the implementations described in this disclosure may be readily apparent to those of ordinary skill in the art, and the generic principles defined herein may be applied to other implementations without departing from the spirit or scope of this disclosure. Thus, the claims are not intended to be limited to the implementations shown herein, hut are to be accorded the widest scope consistent with this disclosure, the principles and the novel features disclosed herein.

Certain features may be described above as acting in certain combinations and even initially claimed as such, one or more features from a claimed combination can in some cases be excised from the combination, and the claimed combination may be directed to a sub-combination or variation of a sub-combination.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Likewise, the word “connected”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or” in reference to a list of two or more items, that word covers all of the following interpretations of the word: any of the items in the list, all of the items in the list, and any combination of the items in the list.

Moreover, conditional language used herein, such as, among others, “may,” “could,” “might,” “can,” “e.g..” “for example.” “such as” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Unless the context clearly requires otherwise, throughout the specifications including the summary, the description and the claims, when reference is made to the tampon apparatus including the tampon and tampon-withdrawal-string., where the tampon is inserted into the vagina of a user, the words “distal” and “proximal” is described with reference to the vaginal opening.

The teachings provided herein can be applied to other systems, not only the systems described herein. The elements and acts of the various embodiments described above can be combined to provide further embodiments.

The tampon apparatus of the present disclosure includes an ordinary tampon, such as a common cylindrical tampon, and a tampon-withdrawal-string, wherein one end of the tampon-withdrawal-string is a tied end, being securely attached to the proximal end of the tampon, and wherein the string, while at a rest state, is arranged in a compact form and as much as possible in an invisible location.

The tampon apparatus can be used with the compact form of the tampon-withdrawal-string, which has the advantage that during usage, the tampon-withdrawal-string in its compact form is barely visible, if at all, and gets less wet and/or dirty, if at all.

The present disclosure proposes two main embodiments of the tampon apparatus, in one embodiment, the tampon-withdrawal-string remains in a compact state (also referred to as a “rest state”) as long as no pulling force is applied on the tampon-withdrawal-string, typically, but not limited to, on the string part located outside the vaginal opening. Typically, the length of the tampon-withdrawal-string in the compact state is shorter than the tampon-withdrawal-string length of an ordinary tampon.

In this embodiment at least a portion of the tampon-withdrawal-string is elastic. Therefore, when pulling the tampon-withdrawal-string (e.g. in order to pull the tampon out of the vaginal opening) the effective length of the tampon-withdrawal-string becomes longer (e.g. when pulling a coiled string its effective length grows), because it is easier for the user to grip and handle a longer tampon-withdrawal-string. At some point, the elastic portion of the tampon-withdrawal-string reaches its longest state, without pulling the tampon itself. This length is referred to as the maximum straightened length D^{S}, which is reached when applying a pulling force F_{P1} ≤F_{S} on the tampon-withdrawal-string.

When releasing any of the pulling force, the form of the tampon-withdrawal-string returns to a compact state (e.g. as much as possible similar to its initial rest state), and the effective length of the tampon-withdrawal-string becomes shorter until reaching the rest state. When pulling the tampon-withdrawal-string with a force F_{P2}>F_{S}, at some point the tampon is being pulled out of the vaginal opening.

The elasticity of the tampon-withdrawal-string can be achieved by manufacturing the tampon-withdrawal-string from various known in the art optional materials, techniques and technologies. For example, the elasticity of the tampon-withdrawal-string can be achieved by using elastic materials (e.g. silicon, polyester); by applying knitting methods that generate elasticity to threads; by twisting yarn; by knitting the yarn in a compact and elastic form with memory like a spiral shape, a zigzag, or as a skein; by shaping the yarn in a compact form and applying different types of coating to the yarn; by using an elastomeric thread as the core and wrapping it with a non-stretch textile fiber; or by producing the yarn using the pneumatic looping technique.

In a second embodiment of the present disclosure, at least a portion of the tampon-withdrawal-string is made of semi-rigid, pliant material that enables the user to arrange the tampon-withdrawal-string in many different desired and convenient shapes, in order, for example without limitations, to facilitate placing the tampon-withdrawal-string hidden from occasional observers (for example without limitations, up and back along her butt crack, or up and forth along her vaginal opening). After the user finishes arranging the tampon-withdrawal-string, the shape of the pliant portion of the tampon-withdrawal-string is preserved as arranged by the user. In the second embodiment of the present disclosure, the effective length of the tampon-withdrawal-string does not necessarily grow when applying a pulling force on the tampon-withdrawal-string (typically, but not limited to, on the string part located outside the vaginal opening). In this embodiment the user can change the form and the direction of the tampon-withdrawal-string at any time, e.g. in order to pull the tampon out of her vaginal opening.

The tampon-withdrawal-string in the second embodiment of the present disclosure can be made of several materials, techniques and technologies.

In summary, the present disclosure discloses a tampon apparatus that includes a tampon having distal, insertion end and a proximal end, and a tampon-withdrawal-string having a free end and a tied end that is securely attached to the proximal end of the tampon.

At least a portion of the tampon-withdrawal-string is a devised-portion, wherein the devised-portion is either elastic or pliant, While the tampon is in an inserted position, the devised-portion facilitates the user to manipulate the tampon-withdrawal-string, or a portion thereof. The free end of the tampon-withdrawal-string is comfortably reachable to a user when the tampon is in an inserted position.

The manipulating of the tampon-withdrawal-string may include straitening the tampon withdrawal string (for example with no limitations, before pulling the tampon out of the vaginal opening). The manipulating of the tampon-withdrawal-string may further include maintaining the tampon-withdrawal-string in a comfort position. The manipulating of the tampon-withdrawal-string may further include maintaining the tampon-withdrawal-string in a mostly invisible position. The manipulating of the tampon-withdrawal-string may further include maintaining the tampon withdrawal string in a position far from wetness/dirt sources in order to make the tampon-withdrawal-string more hygienic. The manipulating of the tampon-withdrawal-string may further include pulling out and rearranging portions of the tampon-withdrawal-string, with or without pulling the tampon from the inserted position.

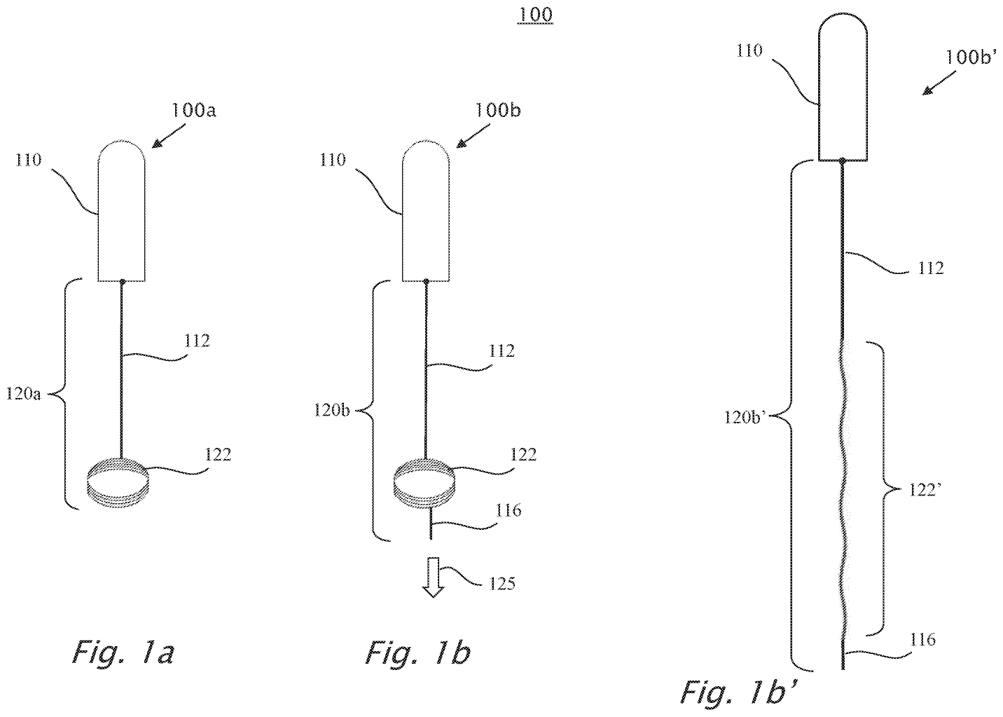

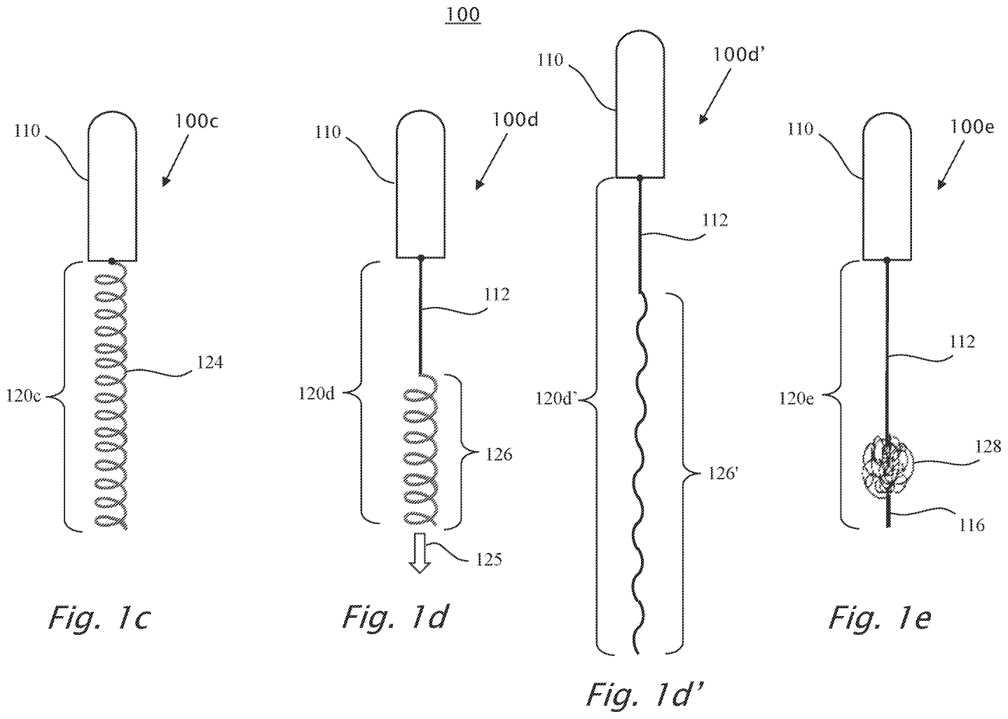

Reference is now made to the drawings. FIG. 1aschematically illustrates an example tampon apparatus 100a, including a common cylindrical tampon 110 and a tampon-withdrawal-string 120a, according to aspects of the present disclosure, wherein the tampon-withdrawal-string 120a is in a rest state. Tampon-withdrawal-string 120a includes a common non-elastic string segment 112 and an elastic, three-dimensional (31D), compact coiled tampon-withdrawal-string devised-portion 122.

FIG. 1b schematically illustrates an example tampon apparatus 100b, including a common cylindrical tampon 110 and a tampon-withdrawal-string 120b, according to aspects of the present disclosure, wherein the tampon-withdrawal-string 120b is in a rest state. Tampon-withdrawal-string 120b includes a common non-elastic string segment 112, an elastic 31) compact coiled tampon-withdrawal-string devised-portion 122 and a pulling tail 116. The pulling tail 116 may ease the grabbing of the free end of tampon-withdrawal-string 120b and pulling out tampon-withdrawal-string 120b, when so desired by the user. It should be appreciated that pulling tail 116 may be pliant.

FIG. 1b′ schematically illustrates an example tampon apparatus 100b′ as in FIG. 1b. wherein the tampon-withdrawal-string 120b′ is shown in a straightened state. While pulling of the pulling tail 116 of tampon-withdrawal-string 120b in direction 125 (see Fig, 1b) the devised-portion 122 straightens and extends in length up to the maximum straightened length, being the straightened state, as illustrated in devised-portion 122′ in

FIG. 1b′. When releasing the pulling force tampon-withdrawal-string 120b′ returns to the rest state, having the shorter effective length (120b).

FIG. 1c schematically illustrates another example tampon apparatus 100e, according to aspects of the present disclosure. Tampon apparatus 100c includes a common cylindrical tampon 110 and a tampon-withdrawal-string 120e, wherein tampon-withdrawal-string 120c is shown in a rest state. Tampon-withdrawal-string 120e includes an elastic 3D helical tampon-withdrawal-string 124, being the devised-portion.

FIG. 1d schematically illustrates an example tampon apparatus 100d, according to aspects of the present disclosure. Tampon apparatus 100d includes a common cylindrical tampon 110 and a tampon-withdrawal-string 120d shown in a rest state.

Tampon-withdrawal-string 120d includes a common non-elastic string segment 112 and an elastic 3D helical tampon-withdrawal-string 126, being the devised-portion.

FIG. 1d′ schematically illustrates an example tampon apparatus 100d′ as in FIG. id, wherein the tampon-withdrawal-string 120d′ is shown in a straightened state. While pulling of the proximal section of the devised-portion 126 of tampon-withdrawal-string 120d in direction 125 (see FIG. 1d) the devised-portion 126 straightens and extends in length up to the maximum straightened state length, as illustrated by devised-portion 126′ in FIG. 1d′. When releasing the pulling force tampon-withdrawal-string 120d′ returns to the rest state, having the shorter effective length (120d).

FIG. 1e schematically illustrates an example tampon apparatus 100e, according to aspects of the present disclosure. Tampon apparatus 100e includes a common cylindrical tampon 110 and a tampon-withdrawal-string 120e shown in a rest state. Tampon-withdrawal-string 120e includes a common non-elastic string segment 112 and an elastic 3D skein segment devised-portion 128 and a pulling tail 116.

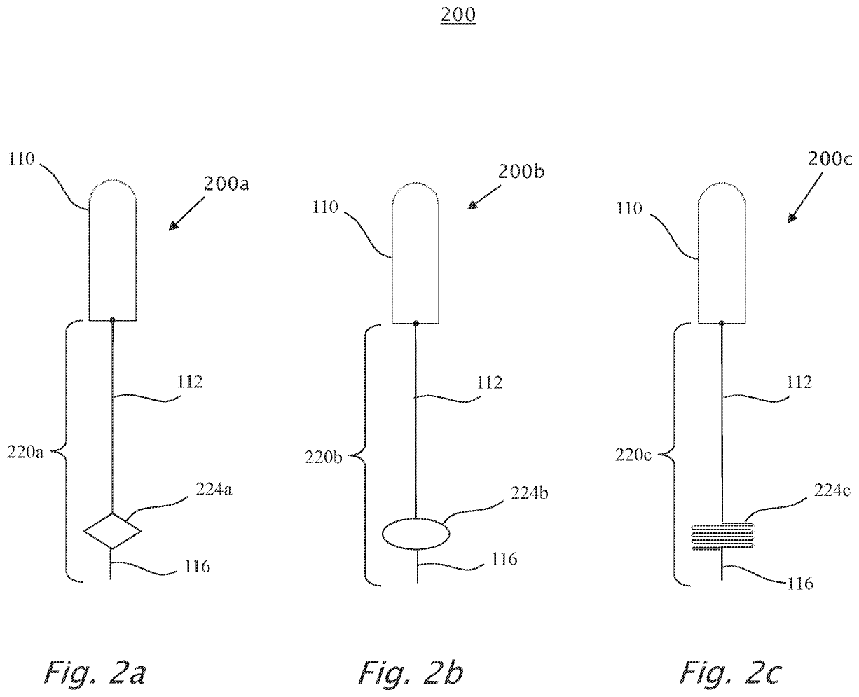

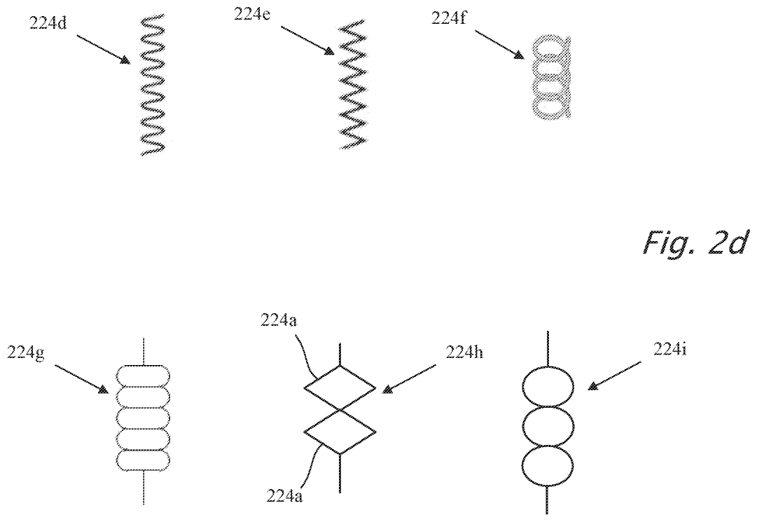

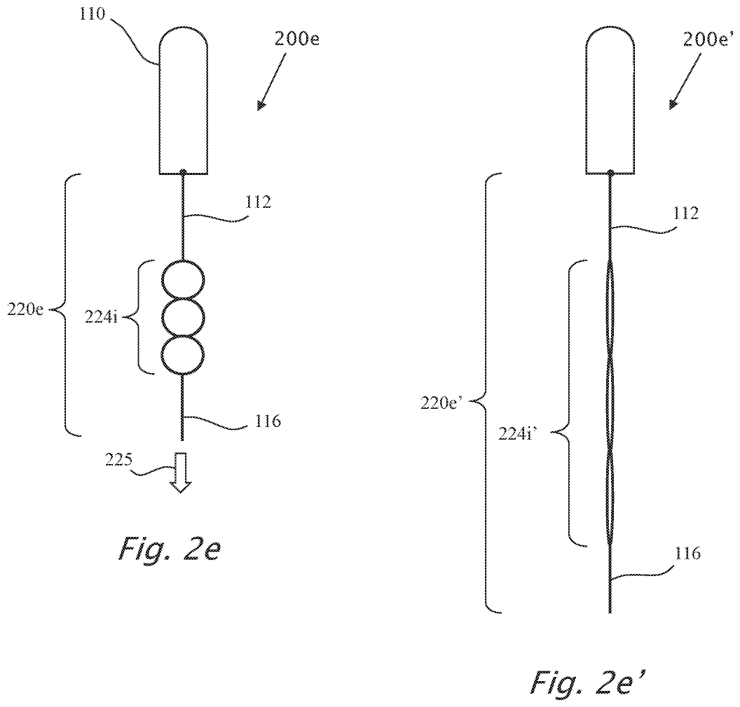

FIG. 2a schematically illustrates an example tampon apparatus 200a, including a common cylindrical tampon 110 and a tampon-withdrawal-string 220a, according to aspects of the present disclosure, wherein the tampon-withdrawal-string 220ais in a rest state. Tampon-withdrawal-string 220aincludes a common non-elastic string segment 112, an elastic two-dimensional (2D) rhombus segment devised-portion 224a and a pulling tail 116.

FIG. 2b schematically illustrates another example tampon apparatus 200b, including a common cylindrical tampon 110 and a tampon-withdrawal-string 220b, according to aspects of the present disclosure, wherein the tampon-withdrawal-string 220b is in a rest state. Tampon-withdrawal-string 220b includes a common non-elastic string segment 112, an elastic 2D rounded loop segment 224b as the devised-portion, and a pulling tail 116.

FIG. 2c schematically illustrates another example tampon apparatus 200c, including a common cylindrical tampon 110 and a tampon-withdrawal-sting 220c, according to aspects of the present disclosure, wherein the tampon-withdrawal-string 220e is in a rest state. Tampon-withdrawal-string 220c includes a common non-elastic string segment 112, an elastic 21) wide zigzag segment 224c with rounded corners as the devised-portion 224c, and a pulling tail 116.

FIG. 2d schematically illustrates more example tampon-withdrawal-strings for a tampon apparatus having various 2D shapes (224d, 224e, 224f, 224g, 224h, 224i) in a rest state, according to aspects of the present disclosure.

FIG. 2e schematically illustrates an example tampon apparatus 200e, according to aspects of the present disclosure. Tampon apparatus 200e includes a common cylindrical tampon 110 and a tampon-withdrawal-string 220e shown in a rest state. Tampon-withdrawal-string 220e includes a common non-elastic string segment 112, a row of 3 elastic 2D rounded loops 224i as the devised-portion, and a pulling tail 116.

FIG. 2e′ schematically illustrates an example tampon apparatus 200e′ as in FIG.

2e, wherein the tampon-withdrawal-string 220e is shown in a straightened state. While pulling of the pulling tail 116 of tampon-withdrawal-string 220e in direction 225 (see FIG. 2e) the devised-portion 224i straightens and extends in length up to the maximum straightened state length, as illustrated by devised-portion 224i′ in FIG. 2e′. When releasing the pulling force tampon-withdrawal-string 220e′ returns to the rest state, having the shorter effective length (220e).

### EXAMPLES

Currently, the withdrawal string of many tampons in the market (namely: a “regular string”) has an almost straight form when in use and at rest state, and its length extends only slightly (negligibly) when the string is being pulled out.

In a first non-limiting example, with reference to Figs. 1b and 1b′, the tampon-withdrawal-string 120b having a coiled devised-portion 122 that is organized in a compact, rest state, having an initial rest length. The tampon-withdrawal-string 120b can be extended in length when a pulling force is applied.

In this example, at a rest state, the effective length of tampon-withdrawal-string 120b can be designed to be shorter than that of a regular withdrawal string. If, for example, the effective length of a regular withdrawal string at a rest state is 12 cm, then the effective length of tampon-withdrawal-string 120b can be, for example, 8 cm, which is 4 cm shorter than the regular tampon withdrawal string length.

When applying a pulling force to the coiled devised-portion 122 in direction 125, the effective length of the compact string 120b is extended. example, in FIG. 1b, if the effective length of the tampon-withdrawal-string 120b at a rest state is 8 cm, and the compact coiled tampon-withdrawal-string devised-portion 122 has 4 loops, each having a diameter of 1 cm, then the effective length of the extended tampon-withdrawal-string 120b′ at a straightened state (see FIG. 1b′) can reach a length of about 20 cm, with no limitations.

In another example, with reference to FIGS. 2e and 2e′, if the effective length of the tampon-withdrawal-string 220e at rest state is S cm, and the compact tampon-withdrawal-string devised-portion 224i has three elastic 2D rounded loops, each having a diameter of 1 cm, then the effective length of the extended tampon-withdrawal-string 220e′ at a straightened state (see withdrawal string segment 224i′ in FIG. 2e′) can reach a length of about 9.7 cm.

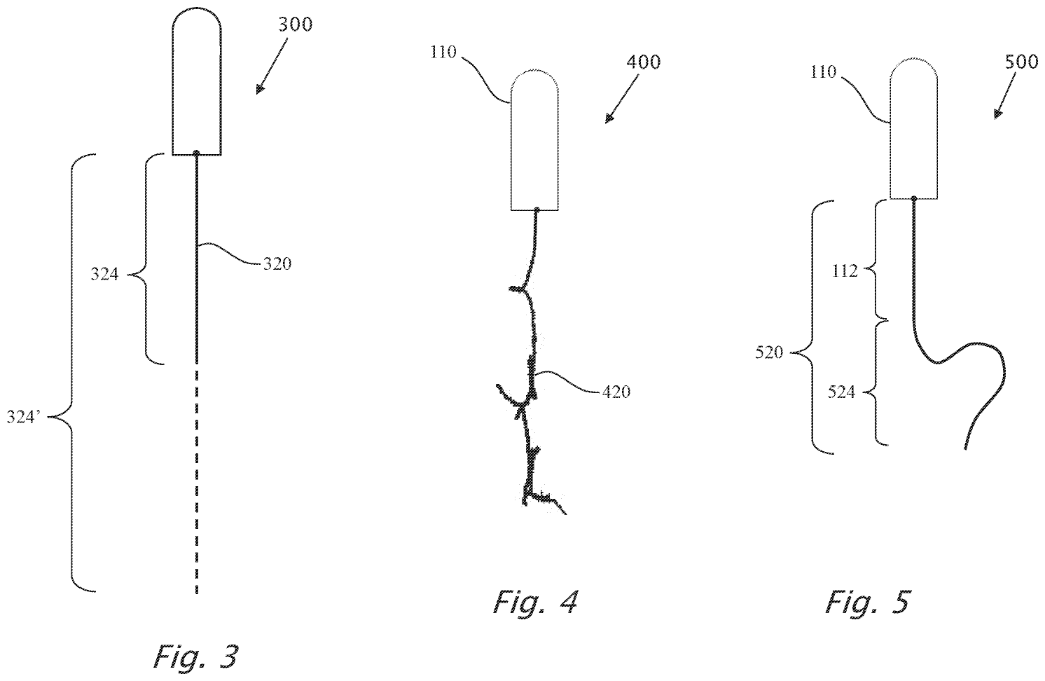

FIG. 3 schematically illustrates an example tampon apparatus 300 having an elastic tampon-withdrawal-string 320, according to aspects of the present disclosure, wherein the solid line segment 324 illustrates an elastic tampon-withdrawal-string (320) in a rest state and the unification of the solid line segment with the segmented line segment 324′ illustrates elastic tampon-withdrawal-string (320) extended up to the maximum length of the straightened state.

FIG. 4 schematically illustrates an example tampon apparatus 400 having a twisted-withdrawal-string 420. according to aspects of the present disclosure, wherein the compact and extendible tampon-withdrawal-string 420 is made of non-elastic material and is shown in a rest state.

It should be appreciated that the compact and extendible devised-portion of a tampon-withdrawal-string can be hidden fully or partially within the vagina, or located outside of the vagina, preferably in proximity as much as possible to the vaginal opening.

FIG. 5 schematically illustrates an example tampon apparatus 500 having a pliant tampon-withdrawal-string 520, according to aspects of the present disclosure, wherein the tampon-withdrawal-string 520 is shown in an example rest state. The pliant tampon-withdrawal-string 520 may include common non-elastic string segment 112, wherein the portion reaching the free end of tampon-withdrawal-string 520, or proximal thereto, is pliant, being the devised-portion 524.

After the user finishes arranging the devised-portion 524 of tampon-withdrawal-string 520, the shape of the pliant devised-portion 524 of the tampon-withdrawal-string 520 is preserved as arranged by the user. In order to pull the tampon 110 out of the vaginal opening, the user can change the form and/or angle of the pliant devised-portion 524 for easier gripping and pulling-out (e.g. shaping the pliant devised-portion 524 in a straight line form with its free end heading to the direction of the floor when the user is standing, or shaping the pliant devised-portion 524 in the form of a hook, or a loop). It is possible to change the form of the pliant devised-portion 524 several times, and after each change the form of the pliant devised-portion 524 remains as last shaped by the user. The pliant devised-portion 524 can be made of, but not limited to, a thin metallic string embraced with a soft material, for example with no limitations, silicon and cotton.

While example materials and manufacturing techniques and technologies for elements have been described, the present disclosure invention is not limited by these materials, techniques and technologies.

Various modifications can be made in the design and operation of the present disclosure invention without departing from its spirit. Thus, while examples of construction of the present disclosure invention have been explained in what is now considered to represent its example embodiments, it should be understood that within the scope of the patent, the present disclosure invention may be practiced otherwise than as specifically illustrated and described.

The features disclosed in the above description and in the drawings may be significant both individually and in any desired combination in order to realize the various embodiments of the present disclosure.

Although the present disclosure invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present disclosure, are contemplated thereby, and are intended to be covered by this patent.

While certain embodiments of the inventions have been described, wherein these embodiments have been presented by way of example only, and are not intended to limit the scope of the disclosure. Indeed, the novel methods and systems described herein may be embodied in a variety of other forms; furthermore, various omissions, substitutions and changes in the form of the methods and systems described herein may be made without departing from the spirit of the disclosure. The accompanying claims and their equivalents are intended to cover such forms or modifications as would fall within the scope and spirit of the disclosure.

1-42. (canceled)

43. A tampon apparatus including a tampon having distal, insertion end and a proximal end, and a tampon-withdrawal-string;
wherein said tampon-withdrawal-string is elastic;
wherein said tampon-withdrawal-string comprises a rest state, which is a compact form of said tampon-withdrawal-string, and a straightened form, which is a longest state that said tampon-withdrawal-string can reach without pulling the tampon itself; said tampon-withdrawal-string is configured to return to said rest state after being released from said straightened form;
wherein, while the tampon is in an inserted position and said tampon-withdrawal-string is in said rest state, said tampon-withdrawal-string has dimensions that keep the tampon-withdrawal-string away from the anus of a user using said tampon apparatus.

44. The tampon apparatus of claim 43, wherein, while the tampon is in an inserted position and said tampon-withdrawal-string is in said rest state, said tampon-withdrawal- string has dimensions that keep the tampon-withdrawal-string away from a urethral opening of a user using said tampon apparatus.

45. The tampon apparatus of claim 43, wherein, while the tampon is in an inserted position and said tampon-withdrawal-string is in said rest state, said tampon-withdrawal-string has dimensions that keep the tampon-withdrawal-string away from pee exiting a urethral opening of a user using said tampon apparatus.

46. The tampon apparatus of claim 43, wherein, while the tampon is in an inserted position and said tampon-withdrawal-string is in said rest state, said tampon-withdrawal-string has dimensions that keep the tampon-withdrawal-string away from feces exiting said anus.

47. The tampon apparatus of claim 43, wherein, while the tampon is in an inserted position and said tampon-withdrawal-string is in said rest state, said tampon-withdrawal-string has dimensions that keep the tampon-withdrawal-string close to a user body and thereby hidden from potential observers.

48. The tampon apparatus of claim 43, wherein a free end of said tampon-withdrawal string is comfortably reachable to a user when the tampon is in said inserted position.

49. The tampon apparatus of claim 43, wherein in said straightened form, said tampon-withdrawal-string extends to a maximum length of DS when pulling said tampon-withdrawal-string with a force FP1<FS, without pulling the tampon from the inserted position, and wherein when pulling said tampon-withdrawal-string with a force FP2>FS, at some point the tampon is being pulled-out of the vaginal opening.

50. The tampon apparatus of claim 43, wherein said tampon-withdrawal-string is arranged in a one-dimensional (1D) form or a two-dimensional (2D) form or a three-dimensional (3D) form.

51. The tampon apparatus of claim 43, wherein said tampon-withdrawal-string comprises one or more of:
a. elastic materials;
b. non-elastic materials.

52. The tampon apparatus of claim 43, wherein elasticity of said tampon-withdrawal-string is formed by applying manufacturing methods selected from the group of methods including:
a. twisting yarn;
b. knitted yarn;
c. knitting the yarn in a compact and elastic form with memory like a spiral shape, a zigzag, or as a skein;
d. shaping the yarn in a compact form and applying different types of coating to the yarn;
e. using one or more elastic threads, possibly wrapped by one or more non-elastic threads;
f. using one or more elastic threads, possibly twisted with one or more non-elastic materials;
g. using one or more elastomeric thread as a core, wherein said core is wrapped with a non-elastic textile fiber; and
h. producing the yarn using the pneumatic looping technique.

53. A method of use by a user of a tampon apparatus comprising a tampon-withdrawal-string, said user having a urethral opening, a vaginal opening of a vagina and an anus, the method comprising:
a. inserting said tampon apparatus in said vaginal opening;
b. maintaining said tampon-withdrawal-string away from the anus while inserted in said vagina;
c. elastically pulling said tampon-withdrawal-string until a length of said tampon-withdrawal-string is comfortable for the purpose of gripping and pulling the tampon apparatus out of the vaginal opening;
d. pulling said tampon-withdrawal-string until said tampon apparatus is pulled out of said vaginal opening.

54. The method according to claim 53, wherein said tampon-withdrawal-string is elastic.

55. The method of claim 53, wherein said tampon-withdrawal-string is in a rest state, which is a compact form of said tampon-withdrawal-string, while said inserting and said maintaining.

56. The method of claim 53, wherein said tampon-withdrawal-string is in a rest state, which is a compact form of said tampon-withdrawal-string, after said inserting and before said elastically pulling.

57. The method of claim 53, wherein said elastically pulling said tampon-withdrawal-string comprises elastically pulling said tampon-withdrawal-string until reaches a straightened form.

58. The method of claim 53, further comprising maintaining said tampon-withdrawal-string away from said urethral opening.

59. The method of claim 53, further comprising maintaining said tampon-withdrawal-string away from pee exiting said urethral opening.

60. The method of claim 53, further comprising maintaining said tampon-withdrawal-string away from feces exiting said anus.

61. The method of claim 53, further comprising maintaining said tampon-withdrawal-string close to the user body and thereby hidden from potential observers.

62. The method of claim 54, wherein said tampon-withdrawal-string performs said maintaining.
